(19) United States
(12) Patent Application Publication
(10) Pub. No.: US 2026/0131072 A1
(43) Pub. Date: May 14, 2026

(54) INTEGRATED CGM AND INSULIN PUMP SYSTEM WITH DIRECT POWER BRIDGE CONNECTION

(71) Applicant: Tandem Diabetes Care, Inc, San Diego, CA (US)

(72) Inventor: Reza Farshchi, San Diego, CA (US)

(21) Appl. No.: 19/379,318

(22) Filed: Nov. 4, 2025

Related U.S. Application Data
(60) Provisional application No. 63/719,429, filed on Nov. 12, 2024.

(51) Int. Cl. A61M 5/142

(52) U.S. Cl. CPC A61M 5/14244; A61M 2202/0007; A61M 2205/3303; A61M 2205/8206; A61M 2209/084

(57) ABSTRACT

Ambulatory infusion pump systems can integrate a CGM sensor with an ambulatory infusion pump via a pump holder or tray that releasably holds the pump and is configured to be worn by a user. A direct electrical connection between the pump and the CGM can be facilitated by a power bridge that provides for power from the pump to the CGM sensor and facilitates communications between the CGM sensor and the pump. By combining the CGM and insulin pump into one integrated system with a direct electrical connection, a more aesthetically pleasing, convenient and reliable system is provided. Diabetes management is simplified by consolidating two essential devices into one.

### PRIORITY CLAIM

The present application claims the benefit of U.S. Provisional Application No. 63/719,429 filed November 12, 2024, which is hereby incorporated herein by reference in its entirety.

### TECHNICAL FIELD

The present disclosure relates generally to ambulatory infusion pumps and, more particularly, to operation of ambulatory infusion pumps in conjunction with a continuous glucose monitor.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Patent Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring devices (CGMs). A CGM device may consist of a sensor placed under the patient’s skin and affixed to the patient via an adhesive patch, a transmitter, and a monitor. A CGM device samples the patient’s interstitial fluid periodically (e.g., once every 1-5 minutes) to estimate blood glucose levels over time. CGMs are advantageous because they provide more frequent insights into a user’s blood glucose levels yet do not require a finger stick each time a reading is taken.

Ambulatory infusion pumps may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

While integrations of CGM devices with insulin pumps provides a number of advantages, the CGM sensor and pump are separately attached to the body resulting in a cumbersome and visually unappealing dual device configuration. Both the pump and the CGM also need to each have a battery, processor, communications element, etc. In addition, in order to establish wireless communications between the devices a pairing procedure must be undertaken. Patients are therefore burdened with managing two distinct systems, each with its own power source and attachment method, leading to increased complexity, higher costs and a less streamlined user experience.

### SUMMARY

Embodiments disclosed herein are directed to ambulatory infusion pump systems that integrate a CGM sensor with an ambulatory infusion pump via a pump holder or tray that releasably holds the pump and is configured to be worn by a user. A direct electrical connection between the pump and the CGM can be facilitated by a power bridge that provides for power from the pump to the CGM sensor and facilitates communications between the CGM sensor and the pump. By combining the CGM and insulin pump into one integrated system with a direct electrical connection, a more aesthetically pleasing, convenient and reliable system is provided. Diabetes management is simplified by consolidating two essential devices into one.

In embodiments, an ambulatory infusion pump system can include an ambulatory infusion pump including a housing, a reservoir configured to contain a medicament, a pumping mechanism configured to deliver the medicament from the reservoir to a user, a battery and an electrical connector on an exterior of the housing. The system can further include a glucose sensor configured to measure a signal indicative of glucose levels of a user. A pump holder can include a tray configured to releasably retain the ambulatory infusion pump and a sensor recess configured to receive the glucose sensor. A power bridge can include a sensor interface and an electrical connector. The power bridge can be configured to be selectively attached to the pump holder with the sensor interface electrically connected to the glucose sensor and the electrical connector interfaced with the electrical connector of the ambulatory infusion pump to facilitate transfer of power from the battery of the ambulatory infusion pump to the glucose sensor for operation of the glucose sensor.

In embodiments, an ambulatory infusion pump system can include an ambulatory infusion pump including a housing, a reservoir configured to contain a medicament, a pumping mechanism configured to deliver the medicament from the reservoir to a user, and a battery. A pump holder can include a tray configured to releasably retain the ambulatory infusion pump, the pump holder including a sensor recess configured to receive a glucose sensor. A power bridge can be configured to be selectively attached to the pump holder to provide a direct electrical connection between the glucose sensor and the ambulatory infusion pump to facilitate transfer of power from the battery of the ambulatory infusion pump to the glucose sensor for operation of the glucose sensor.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 depicts a schematic representation of an infusion pump system according to an embodiment of the disclosure.

FIG. 5 depicts an infusion pump system according to an embodiment of the disclosure.

FIG. 6 depicts the pump holder of the infusion pump system of FIG. 5.

FIG. 7 depicts the CGM sensor of the infusion pump system of FIG. 5.

FIG. 8 depicts a portion of the infusion pump of the infusion pump system of FIG. 5.

FIGS. 9A-9B depict the power bridge of the infusion pump system of FIG. 5.

FIG. 10 depicts the pump holder of the infusion pump system of FIG. 5 with the CGM sensor and an infusion cannula inserted therein.

FIGS. 11A-11C depict the ambulatory infusion pump system of FIG. 5.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

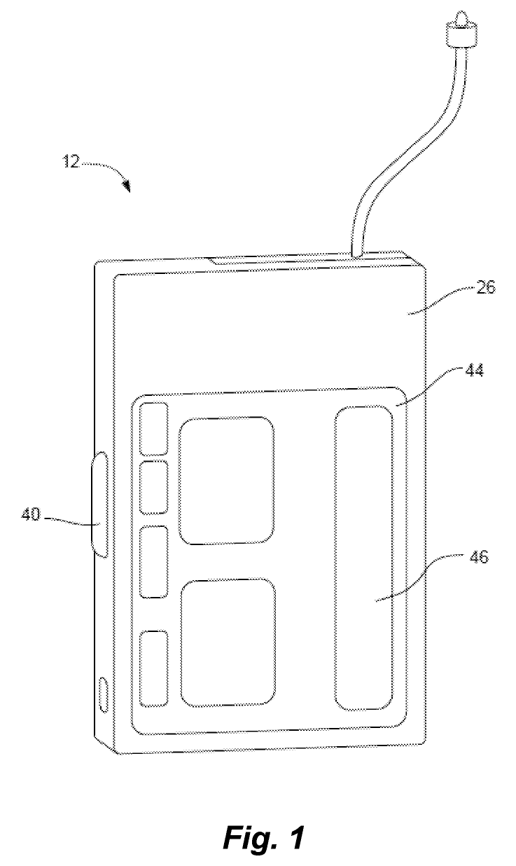

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Patent No. 8,287,495, which is incorporated herein by reference in its entirety. In other embodiments, the medical device can be an infusion pump configured to deliver one or more additional or other medicaments to a patient.

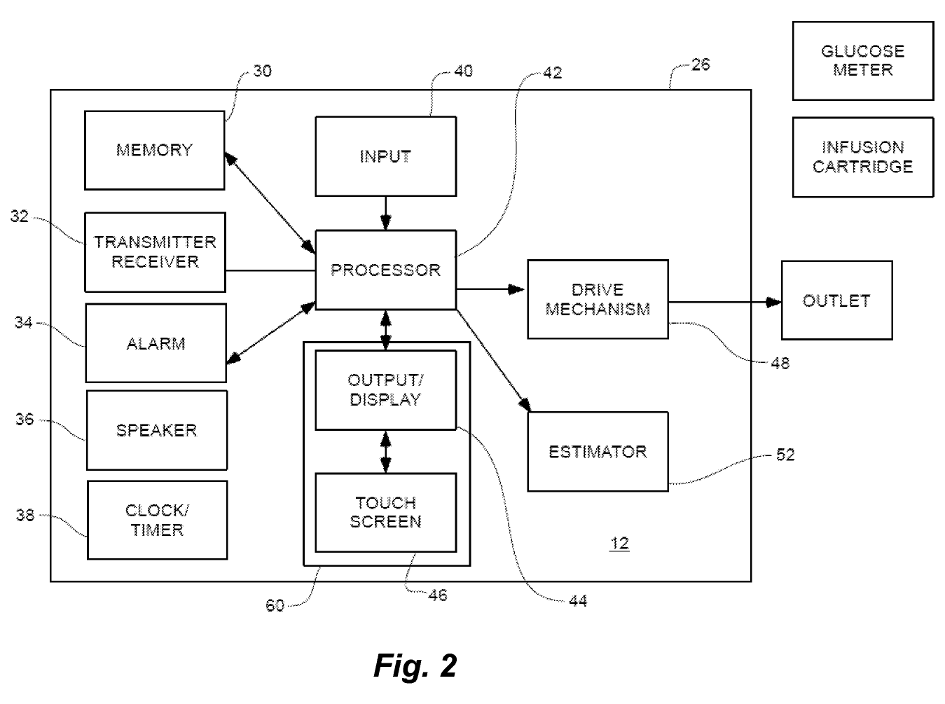

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52, and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor 42 to receive signals and/or other data from one or more input devices, such as sensors that may sense pressure, temperature and/or other parameters.

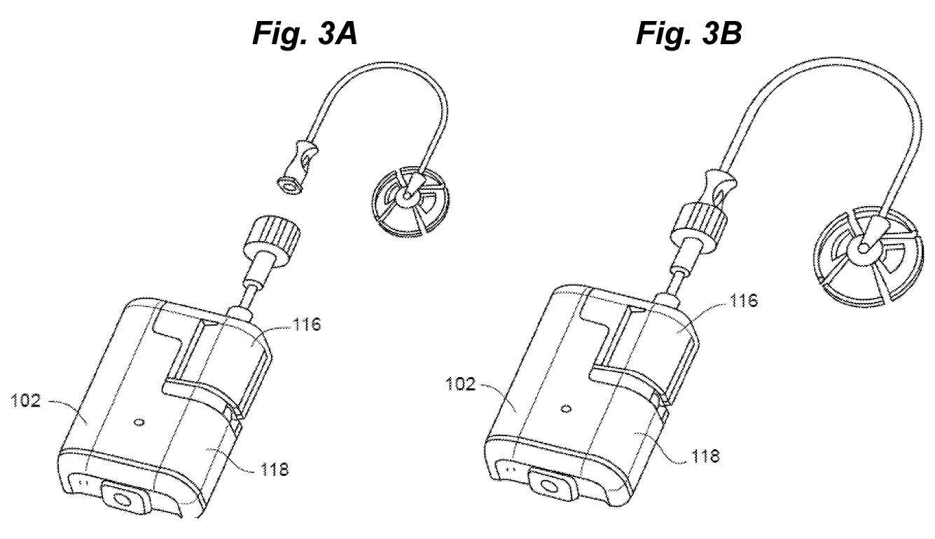

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor 42 that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote device or a consumer electronic device such as a smartphone having a processor executing an application configured to enable the device to transmit operating commands to the processor 42 of pump 102. In some embodiments, processor 42 can also transmit information to one or more separate devices, such as information pertaining to device parameters, alarms, reminders, pump status, etc. Such separate device can include any remote display, remote device, remote control, or a consumer electronic device as described previously.

Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Patent No. 10,279,106 and U.S. Patent Publication Nos. 2016/0339172 and 2017/0049957, each of which is hereby incorporated herein by reference in its entirety.

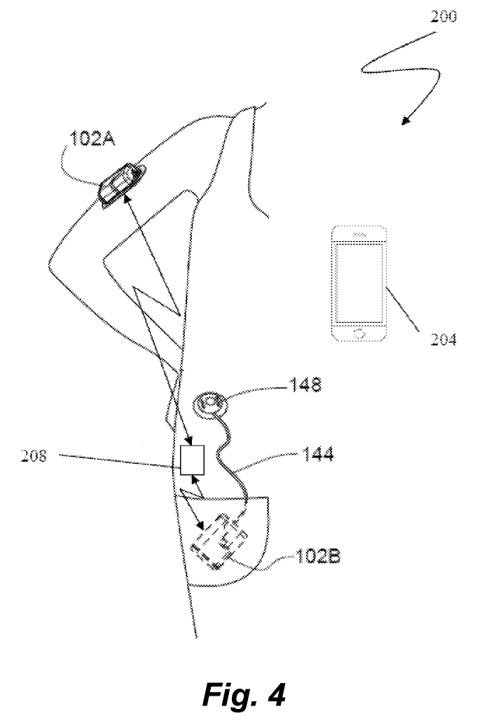

FIG. 4 depicts a schematic representation of a pump system 200 according to an embodiment of the disclosure. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter can communicate that information to pump 102A/B. Pump 208 can then use the glucose data in making therapy determinations. The system can also include one or more devices such as a smartphone 204 or other multi-purpose consumer electronic device capable of operating a software application to communicate with and/or control the pump and, alternatively or additionally, a dedicated remote device designed specifically for use with pump 102A/102B. The smartphone 204 or other remote electronic device can in some embodiments also be capable of communication with CGM sensor/transmitter 208 and can calculate therapy commands for execution by the pump 102A/102B based on the glucose levels.

Although as described above infusion pumps and continuous glucose monitors are separate devices, there are disadvantages to this configuration. For example, the requirement for Bluetooth or other wireless communications between the pump and the CGM require the devices to be paired. This requires the user to undertake a complex pairing procedure to pair the two devices and errors can arise if the devices become unpaired or communication issues arise between the devices. The CGM sensor and the pump also require two different attachment sites and separate insertions into the user’s body. In addition, the separate devices each require their own batteries, processors, communications devices and associated circuitry. Embodiments described herein integrate the CGM with the ambulatory infusion pump to address each of these disadvantages.

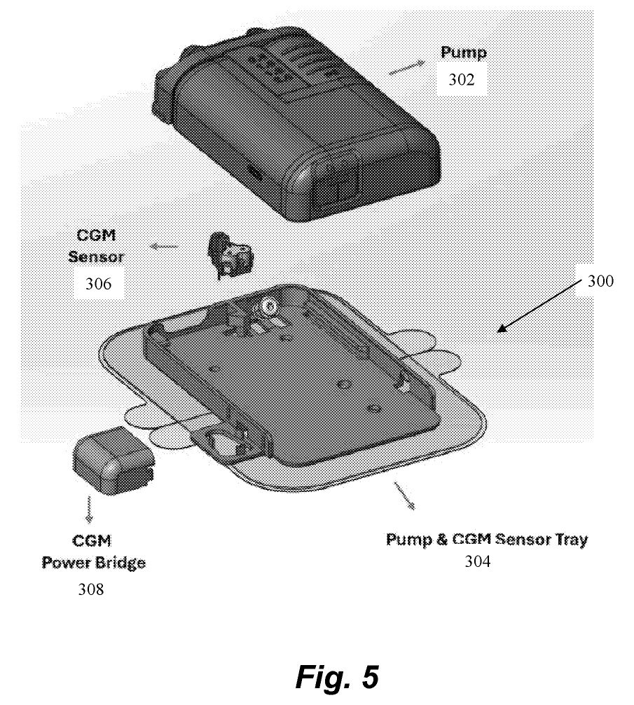

Referring to FIG. 5, an ambulatory infusion pump system 300 according to an embodiment of the disclosure is depicted. Ambulatory infusion pump system 300 is an integrated CGM and insulin pump system that can include a user-wearable infusion pump 302, a pump holder or tray 304 and a CGM sensor 306. A power bridge attachment 308 facilitates the interconnection of the infusion pump 302 and the CGM sensor, as will be described in more detail below.

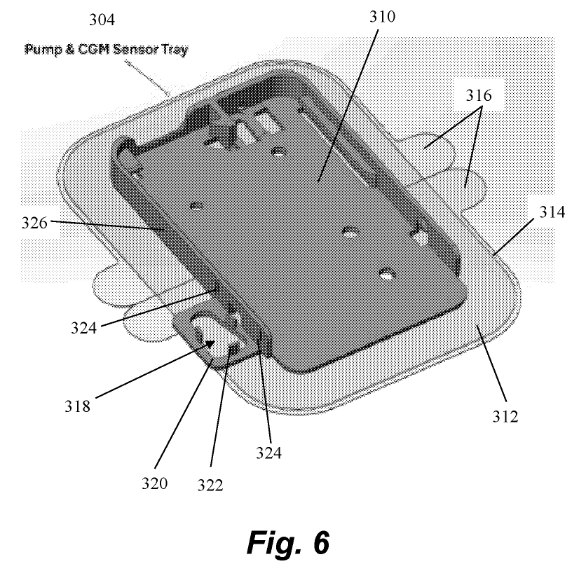

Pump holder 304 is also depicted in FIG. 6. Pump holder 304 can include a tray body 310 attached to an adhesive patch 312 that can releasably attach the pump holder 304 to a body of a user. A patch liner 314 comprising one or more pieces can include tabs 316 for removing patch liner 314 to expose the adhesive underside of adhesive patch 312 for attachment to the user. Tray body 310 can include a number of features related to guiding and releasably retaining an infusion pump on tray body 310, which are not described in detail herein. Further details regarding such features can be found in U.S Patent Publication Nos. 2024/0050650 and 2024/0226423, each of which is hereby incorporated herein by reference. Pump holder 304 can include a sensor recess 318 defined through a tray extension 320 extending outwardly from a perimeter of tray body 310 sized and shaped to receive a CGM sensor such as sensor 306. The size and shape of sensor recess 318 can vary based on the configuration of a particular sensor. Tray extension 320 can include one or more projections 322 configured to aid in retaining a CGM sensor in sensor recess 318. Pump holder 304 can further define a pair of latch apertures 324 in a retaining wall 326 of tray body 310 which aid in retaining the power bridge thereon.

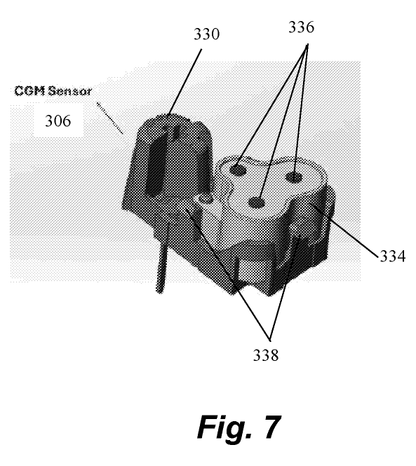

A CGM sensor 306 that can be used with a system 300 as disclosed herein is depicted in FIG. 7 herein. CGM sensor 306 can include a sensor housing 330 with a sensor probe 332 extending from the housing that is configured to be inserted into the body of the user. A sensor body 334 can include a plurality of electrical contacts 336. Although three electrical contacts 336 are depicted, greater or fewer electrical contacts can be employed. The particular geometries and size of sensor housing 330 and sensor body 334 are exemplary only and system can be adapted as needed for various other applications. In the depicted embodiment, sensor housing 330 includes a plurality of outwardly extending ledges 338 that interface with the projections 322 on the tray extension 320 of pump holder 304 to aid in retaining CGM sensor 304 in sensor recess 318.

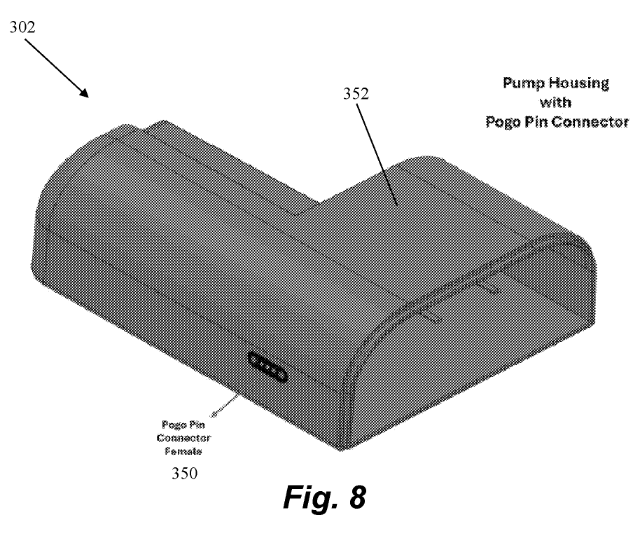

FIG. 8 depicts a portion of an ambulatory infusion pump 302 for use in an ambulatory infusion pump system 300 according to the disclosure. Ambulatory infusion pump 302 can include an electrical connector 350 on an exterior surface of a housing 352 of pump 302. In the depicted embodiment, electrical connector 350 comprises a plurality of spring-loaded pogo pins 354 (depicted as the female pogo connector). In various other embodiments, other known types of electrical connections can be employed. Additional circuitry can electrically couple the electrical connector 350 with the internal electrical components of the pump 302, such as the battery and processor.

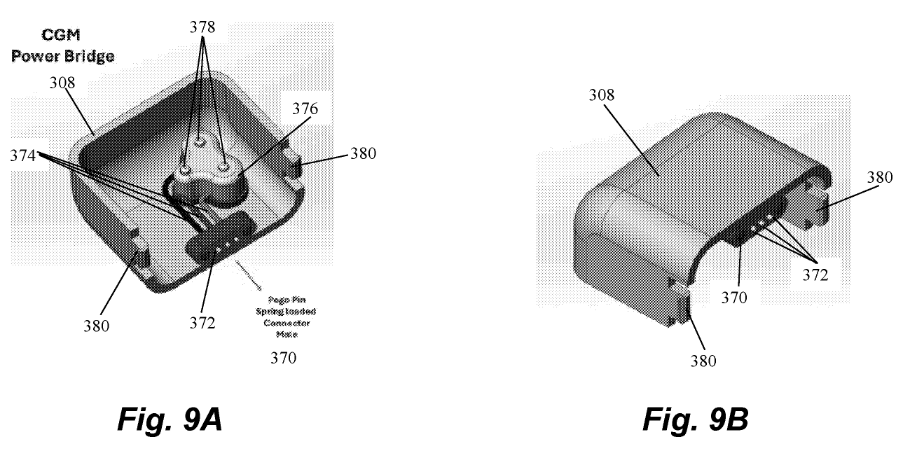

FIG. 9A-9B depict the power bridge 308 of ambulatory infusion pump system 300 that electrically connects the CGM sensor 306 with the pump 202. Power bridge 308 includes an electrical connector 370 that interfaces with electrical connector 350 on housing 352 of pump 302. In this embodiment, electrical connector 370 includes a plurality of pogo pins 372 (e.g., male connector) that mate with the pogo pins 354 on the electrical connector 350 on pump. Electrical connectors 350, 370 can further include complementary magnets that aid in maintaining the connectors together. Electrical connector 370 can be connected with a plurality of wires 374 to sensor interface 376. Sensor interface 376 can have a plurality of electrical contacts 378 that contact and electrically connect to electrical contacts 336 on CGM sensor 306. In the depicted embodiment, sensor interface 376 includes three electrical contacts 378 corresponding to the three electrical contacts 336 of CGM sensor 306, with a corresponding wire 374 for each, but greater or fewer electrical contacts and/or wires can be present. Power bridge 308 can further include a pair of latches 380 configured to provide a snap-fit with latch apertures 324 in pump holder 304 to releasably connect power bridge 308 to pump holder 304, but any other type of mechanical connection can alternatively be employed.

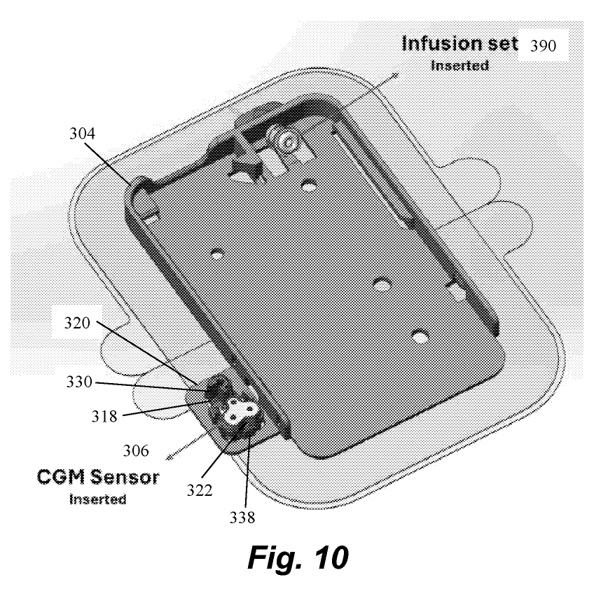

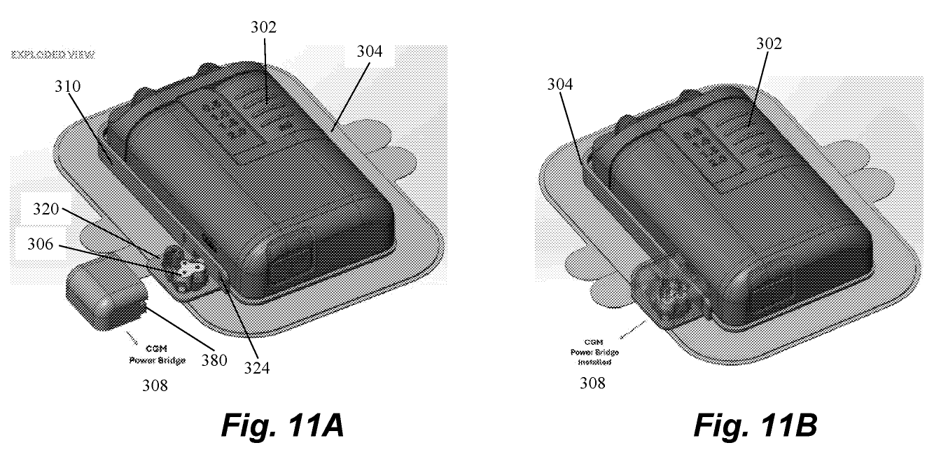

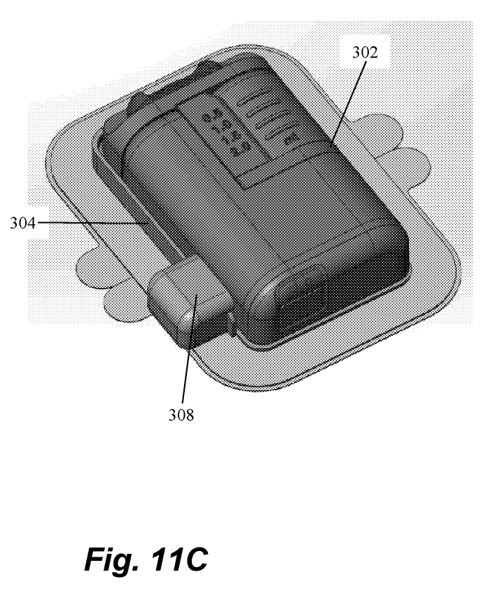

FIG. 10 depicts the pump holder 304 of ambulatory infusion pump system 300 with the CGM sensor 306 and an infusion cannula 390 inserted through pump holder 304 (and into a user’s body). CGM sensor 306 nests within sensor aperture 318 of tray extension 320 with projections 322 on tray extension 320 interfacing with ledges 338 on the housing 330 of CGM sensor 306 to retain the CGM sensor 306 thereon. Pump 302 can then be releasably attached to the tray body 310 of pump holder 304 as shown in FIG. 11A. Power bridge 308 is then slid over tray extension 320 and releasably attached to tray body 310 be inserting latches 380 of power bridge 308 into latch apertures 324 in retaining wall 326 of tray body 310 connected to tray extension as shown in FIGS. 11A-11C. In doing so, the electrical contacts 378 of sensor interface 376 of power bridge 308 are brought into contact with the electrical contacts 336 of CGM sensor 306 and the electrical connector 370 of power bridge 308 is mated with the electrical connector 350 on the outer housing 352 of the pump. The creates an electrical coupling from the CGM sensor 306 to the internal processor of the pump 302 via the sensor interface 376, corresponding electrical connectors 350, 370 in the power bridge 308 and pump 302 and internal circuitry in the pump 302.

In embodiments disclosed herein, a CGM sensor 306 is therefore powered through a direct electrical connection with an insulin pump 302 via power bridge 308 such that the CGM sensor receives power from the internal battery in the pump 302. This eliminates the need for a traditional battery within the CGM sensor, reducing its size and cost. Elimination of the battery further eliminates the need for replacing the battery in the sensor, simplifying device maintenance and enhancing reliability since there will be no gaps in sensor readings due to a dead battery. Power bridge 308 is particularly advantageous in providing an ability to provide a direct power connection between a CGM sensor and an insulin pump in a circumstance where the physical footprint and configuration of the CGM sensor makes it impractical to directly physically interface the CGM sensor with the pump.

The insulin pump 302 and CGM sensor 306 are both retained by the pump holder of the pump holder in a manner that ensures power transfer between the pump and the sensor while maintaining a sleek, unified appearance on the body. This reduces the number of separate devices on the body, providing a more discreet and visually appealing system.

In these embodiments, communications between the CGM sensor and pump can also be accomplished via the direct electrical connection facilitated by power bridge. Pump can include internal circuitry to transmit the signals measured by the CGM sensor to the pump processor. The processor of pump can include programming sufficient to receive the CGM signals and to process those signals for use of the glucose levels indicated by the signals in providing therapy with the pump. In such embodiments, no Bluetooth or other wireless communications between the devices is required because the CGM signals are sent to the pump utilizing the direct electrical connection between the CGM sensor and the pump. As such, the CGM does not require a complex pairing procedure and wireless connectivity issues preventing transmission of CGM data will not occur. There are therefore further cost savings in the system because the CGM also does not need a processor, wireless communications elements and associated circuitry. The battery life of the pump will also be enhanced due to reduced power consumption from not having continual wireless communications with the CGM, because the power consumption of providing power to the CGM is significantly lower that the power required for wireless communications with the CGM.

In some embodiments, data obtained by the CGM sensor can be stored within a memory of the pump. This data could then be periodically synced to a smartphone application, the cloud or another external source to which the pump can periodically be connected. This can enhance data accessibility and historical data tracking, analysis and trends for patients and/or healthcare providers.

In embodiments, the pump will be able to determine if the connection with the CGM sensor has been disconnected because the pump will no longer be receiving data from the CGM and/or if the CGM loses power. In such circumstances, the pump can provide an alert informing the user that the CGM has become disconnected and/or has low or no power.

In some embodiments, the system can include a dual-purpose inserter (not pictured) to optionally allow for simultaneous insertion of both the CGM sensor and the insulin cannula for the pump into the body. This can simplify the insertion process for patients. Separate inserters can also be used. This may be beneficial in situations where the CGM sensor needs to be replaced more frequently than the cannula, or vice versa.

In embodiments, an ambulatory infusion pump system can include an ambulatory infusion pump including a housing, a reservoir configured to contain a medicament, a pumping mechanism configured to deliver the medicament from the reservoir to a user, a battery and an electrical connector on an exterior of the housing. The system can further include a glucose sensor configured to measure a signal indicative of glucose levels of a user. A pump holder can include a tray configured to releasably retain the ambulatory infusion pump and a sensor recess configured to receive the glucose sensor. A power bridge can include a sensor interface and an electrical connector. The power bridge can be configured to be selectively attached to the pump holder with the sensor interface electrically connected to the glucose sensor and the electrical connector interfaced with the electrical connector of the ambulatory infusion pump to facilitate transfer of power from the battery of the ambulatory infusion pump to the glucose sensor for operation of the glucose sensor.

In some embodiments, the tray includes a tray body and a tray extension extending outwardly from a perimeter of the tray body, and wherein the sensor recess is formed through the tray extension.

In some embodiments, the tray extension extends outwardly from a side of the tray body.

In some embodiments, the tray extension comprises one or more projections extending upwardly from the sensor recess to aid in retaining the glucose sensor in the sensor recess.

In some embodiments, the sensor recess has a shape generally matching a shape of a perimeter of the glucose sensor.

In some embodiments, the power bridge comprises a housing configured to be selectively attached to the tray.

In some embodiments, the housing includes a pair of latches selectively attachable to a corresponding pair of apertures in the tray.

In some embodiments, the sensor interface and the electrical connection of the power bridge are mounted to an interior of the housing.

In some embodiments, the electrical connector on the exterior of the housing of the ambulatory infusion pump and the electrical connector of the power bridge each comprise a plurality of pogo pins.

In some embodiments, the glucose sensor does not include an internal battery.

In embodiments, an ambulatory infusion pump system can include an ambulatory infusion pump including a housing, a reservoir configured to contain a medicament, a pumping mechanism configured to deliver the medicament from the reservoir to a user, and a battery. A pump holder can include a tray configured to releasably retain the ambulatory infusion pump, the pump holder including a sensor recess configured to receive a glucose sensor. A power bridge can be configured to be selectively attached to the pump holder to provide a direct electrical connection between the glucose sensor and the ambulatory infusion pump to facilitate transfer of power from the battery of the ambulatory infusion pump to the glucose sensor for operation of the glucose sensor.

In some embodiments, the tray includes a tray body and a tray extension extending outwardly from a perimeter of the tray body, and wherein the sensor recess is formed through the tray extension.

In some embodiments, the tray extension extends outwardly from a side of the tray body.

In some embodiments, the tray extension comprises one or more projections extending upwardly from the sensor recess to aid in retaining the glucose sensor in the sensor recess.

In some embodiments, the sensor recess has a shape generally matching a shape of a perimeter of the glucose sensor.

In some embodiments, the power bridge comprises a housing configured to be selectively attached to the tray.

In some embodiments, the housing includes a pair of latches selectively attachable to a corresponding pair of apertures in the tray.

In some embodiments, a sensor interface configured to electrically connect to the glucose sensor and an electrical connection configured to electrically connect to the ambulatory infusion pump are mounted to an interior of the housing of the power bridge.

In some embodiments, the direct electrical connection between the glucose sensor and the ambulatory infusion pump is provided by connecting an electrical connector of the power bridge to an electrical connector on the exterior of the housing of the ambulatory infusion pump.

In some embodiments, the glucose sensor does not include an internal battery.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to producenumerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Also incorporated herein by reference in their entirety are commonly owned U.S. Patent Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; 12,214,159; and 12,357,751 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; ; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. Patent Applications Nos. 17/368,968; 17/896,492; 18/398,543; 18/962,169; 19/003,140; 19/003,164; 19/119,554; 19/134,333; 19/205,083; 19/220,426; 19/221,933; 19/225,150; and 19/252,256.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. An ambulatory infusion pump system, comprising:
an ambulatory infusion pump including a housing, a reservoir configured to contain a medicament, a pumping mechanism configured to deliver the medicament from the reservoir to a user, a battery, and an electrical connector on an exterior of the housing;
a pump holder including a tray configured to releasably retain the ambulatory infusion pump, the pump holder including a sensor recess configured to receive a glucose sensor; and
a power bridge including a sensor interface and an electrical connector, the power bridge configured to be selectively attached to the pump holder with the sensor interface electrically connected to the glucose sensor in the sensor recess and the electrical connector interfaced with the electrical connector of the ambulatory infusion pump to facilitate transfer of power from the battery of the ambulatory infusion pump to the glucose sensor for operation of the glucose sensor.

2. The ambulatory infusion pump system of claim 1, wherein the tray includes a tray body and a tray extension extending outwardly from a perimeter of the tray body, and wherein the sensor recess is formed through the tray extension.

3. The ambulatory infusion pump system of claim 2, wherein the tray extension extends outwardly from a side of the tray body.

4. The ambulatory infusion pump system of claim 2, wherein the tray extension comprises one or more projections extending upwardly from the sensor recess to aid in retaining the glucose sensor in the sensor recess.

5. The ambulatory infusion pump system of claim 1, wherein the sensor recess has a shape generally matching a shape of a perimeter of the glucose sensor.

6. The ambulatory infusion pump system of claim 1, wherein the power bridge comprises a housing configured to be selectively attached to the tray.

7. The ambulatory infusion pump system of claim 6, wherein the housing includes a pair of latches selectively attachable to a corresponding pair of apertures in the tray.

8. The ambulatory infusion pump system of claim 6, wherein the sensor interface and the electrical connection of the power bridge are mounted to an interior of the housing.

9. The ambulatory infusion pump system of claim 1, wherein the electrical connector on the exterior of the housing of the ambulatory infusion pump and the electrical connector of the power bridge each comprise a plurality of pogo pins.

10. The ambulatory infusion pump system of claim 1, wherein the glucose sensor does not include an internal battery.

11. An ambulatory infusion pump system, comprising:
an ambulatory infusion pump including a housing, a reservoir configured to contain a medicament, a pumping mechanism configured to deliver the medicament from the reservoir to a user, and a battery;
a pump holder including a tray configured to releasably retain the ambulatory infusion pump, the pump holder including a sensor recess configured to receive a glucose sensor; and
a power bridge configured to be selectively attached to the pump holder to provide a direct electrical connection between the glucose sensor and the ambulatory infusion pump to facilitate transfer of power from the battery of the ambulatory infusion pump to the glucose sensor for operation of the glucose sensor.

12. The ambulatory infusion pump system of claim 11, wherein the tray includes a tray body and a tray extension extending outwardly from a perimeter of the tray body, and wherein the sensor recess is formed through the tray extension.

13. The ambulatory infusion pump system of claim 12, wherein the tray extension extends outwardly from a side of the tray body.

14. The ambulatory infusion pump system of claim 12, wherein the tray extension comprises one or more projections extending upwardly from the sensor recess to aid in retaining the glucose sensor in the sensor recess.

15. The ambulatory infusion pump system of claim 11, wherein the sensor recess has a shape generally matching a shape of a perimeter of the glucose sensor.

16. The ambulatory infusion pump system of claim 11, wherein the power bridge comprises a housing configured to be selectively attached to the tray.

17. The ambulatory infusion pump system of claim 16, wherein the housing includes a pair of latches selectively attachable to a corresponding pair of apertures in the tray.

18. The ambulatory infusion pump system of claim 16, wherein a sensor interface configured to electrically connect to the glucose sensor and an electrical connection configured to electrically connect to the ambulatory infusion pump are mounted to an interior of the housing of the power bridge.

19. The ambulatory infusion pump system of claim 11, wherein the direct electrical connection between the glucose sensor and the ambulatory infusion pump is provided by connecting an electrical connector of the power bridge to an electrical connector on the exterior of the housing of the ambulatory infusion pump.

20. The ambulatory infusion pump system of claim 11, wherein the glucose sensor does not include an internal battery.
